(19) United States
(12) Patent Application Publication
(10) Pub. No.: US 2016/0150990 A1
(43) Pub. Date: Jun. 2, 2016

(54) PHOTOACOUSTIC APPARATUS, SUBJECT INFORMATION ACQUISITION METHOD, AND PROGRAM

(71) Applicant: CANON KABUSHIKI KAISHA, Tokyo (JP)

(72) Inventors: Minoru Ohkoba, Utsunomiya-shi (JP); Kenichi Nagae, Yokohama-shi (JP); Kazuhito Oka, Tokyo (JP); Takeshi Sekiya, Kawasaki-shi (JP)

(21) Appl. No.: 14/948,113

(22) Filed: Nov. 20, 2015

(30) Foreign Application Priority Data
Nov. 28, 2014 (JP) 2014-242454
Oct. 15, 2015 (JP) 2015-204123

(51) Int. Cl. A61B 5/0456; A61B 5/026; A61B 5/00

(52) U.S. Cl. CPC A61B 5/0456; A61B 5/0095; A61B 5/7278; A61B 5/742; A61B 5/026; A61B 2560/0475

(57) ABSTRACT

A photoacoustic apparatus includes a light irradiation unit configured to irradiate a subject with pulsed light a plurality of times, a reception unit configured to receive photoacoustic waves generated by the plurality of times of irradiation of the subject with the pulsed light from the light irradiation unit, and output a plurality of signals corresponding to the plurality of times of light irradiation, a blood information acquisition unit configured to acquire information on an amount of blood of the subject, and a subject information acquisition unit configured to acquire subject information of a target region in the subject based on the plurality of signals, wherein the subject information acquisition unit acquires the subject information based on a plurality of signals acquired in a common period in each of repeated cycles of fluctuation of the amount of blood.

### BACKGROUND OF THE INVENTION

1. Field of the Invention

The present invention relates to a photoacoustic apparatus using photoacoustic effect.

2. Description of the Related Art

Photoacoustic imaging technology is one of imaging technologies that use light. In photoacoustic imaging, first, a subject is irradiated with pulsed light generated by a light source. The irradiation light is propagated and diffused in the subject and absorbed at a plurality of portions in the subject to generate a photoacoustic wave. A transducer converts the photoacoustic wave into an electrical signal, and a processing apparatus performs analysis processing on the electrical signal to acquire information on an optical characteristic value in the subject.

A generated sound pressure (hereinafter, also referred to as “initial sound pressure”) P₀ of the photoacoustic wave generated from an optical absorber in the subject can be expressed by the following Equation:

P₀=Γ·μₐ·Γ·Φ (1),

where, Γ is a Gruneisen coefficient, which is a quotient of a product of a volume expansion coefficient β and the square of a sound speed c that is divided by a specific heat at constant pressure Cₚ, and Φ is the amount of light at a position (local region) (the amount of light having reached the absorber, which is also referred to as “optical fluence”).

The initial sound pressure P₀ can be calculated using a received signal (photoacoustic (PA) signal) output from a probe having received the photoacoustic wave.

It is known that the value of the Gruneisen coefficient is substantially constant if the tissue is determined. Thus, a product of an optical absorption coefficient μₐ and the amount of light Φ, i.e., optical energy absorption density, can be obtained by measuring and analyzing time changes in PA signals at a plurality of portions.

Japanese Patent Application Laid-Open No. 2013-248077 discusses a photoacoustic image generating apparatus configured to generate photoacoustic images of blood vessels based on photoacoustic waves induced by light.

Meanwhile, in a case where a measurement target is a living organism, a signal obtained by photoacoustic measurement may be affected by pulsation of the living organism. For example, in a case where an optical absorber is hemoglobin, if a signal is acquired at a timing at which the amount of blood in a blood vessel is large, the sound pressure of a generated photoacoustic wave is high according to Equation (1), because the amount of hemoglobin existing in the measured region is large. Thus, it is predicted that the signal-to-noise (S/N) ratio of the obtained signal is comparatively high. On the other hand, if a signal is acquired at a timing at which the amount of blood in the blood vessel is small, the sound pressure of a generated photoacoustic wave is low even if the measured region is the same, because the amount of hemoglobin existing in the measured region is small. Thus, it is predicted that the S/N ratio of the received signal of the photoacoustic wave generated at the timing at which the amount of blood is small is comparatively low. In other words, the accuracy of acquired subject information varies depending on the amount of blood in the blood vessel.

### SUMMARY OF THE INVENTION

According to an aspect of the present invention, a photoacoustic apparatus includes a light irradiation unit configured to irradiate a subject with pulsed light a plurality of times, a reception unit configured to receive photoacoustic waves generated by the plurality of times of irradiation of the subject with the pulsed light from the light irradiation unit, and output a plurality of signals corresponding to the plurality of times of light irradiation, a blood information acquisition unit configured to acquire information on an amount of blood of the subject, and a subject information acquisition unit configured to acquire subject information of a target region in the subject based on the plurality of signals, wherein the subject information acquisition unit acquires the subject information based on a plurality of signals acquired in a common period in each of repeated cycles of fluctuation of the amount of blood.

Further features of the present invention will become apparent from the following description of exemplary embodiments with reference to the attached drawings.

### BRIEF DESCRIPTION OF THE DRAWINGS

FIG. 1 illustrates a configuration of a photoacoustic apparatus according to an exemplary embodiment.

FIG. 2 is a flow chart illustrating a subject information acquisition method according to a first exemplary embodiment.

FIGS. 3A to 3D illustrate various types of sequences according to the first exemplary embodiment.

FIG. 4 is a flow chart illustrating a subject information acquisition method according to a second exemplary embodiment.

FIG. 5 illustrates a display example according to the second exemplary embodiment.

### DESCRIPTION OF THE EMBODIMENTS

Various exemplary embodiments of the present invention will be described in detail below with reference to the drawings. In principle, the same components are given the same reference numeral, and description thereof is omitted.

As described above, the S/N ratio of a received signal of a photoacoustic wave generated in a region where the amount of blood is small is comparatively low. Thus, when a photoacoustic apparatus acquires subject information of a target region, the accuracy of subject information acquired in a period in which the amount of blood is small may be low. In a first exemplary embodiment, the description will be given of an example in which the amount of blood in a subject region is estimated based on an electrocardiographic signal and subject information is acquired based on an acoustic wave generated in a period in which the amount of blood is relatively large.

A photoacoustic apparatus according to the present exemplary embodiment is an apparatus configured to acquire subject information based on a received signal of a photoacoustic wave. The subject information according to the present exemplary embodiment refers to information on a subject that is acquired from a received signal of a photoacoustic wave generated by photoacoustic effect. Specifically, the subject information is generated sound pressure (initial sound pressure), optical energy absorption density, optical absorption coefficient, concentration of a substance that forms the tissue, etc. The concentration of a substance refers to oxygen saturation, oxyhemoglobin concentration, deoxyhemoglobin concentration, total hemoglobin concentration, etc. The total hemoglobin concentration refers to a sum of the oxyhemoglobin concentration and the deoxyhemoglobin concentration. Further, the subject information may be distribution data such as optical absorption coefficient distribution, oxygen saturation distribution, etc.

### Basic Configuration

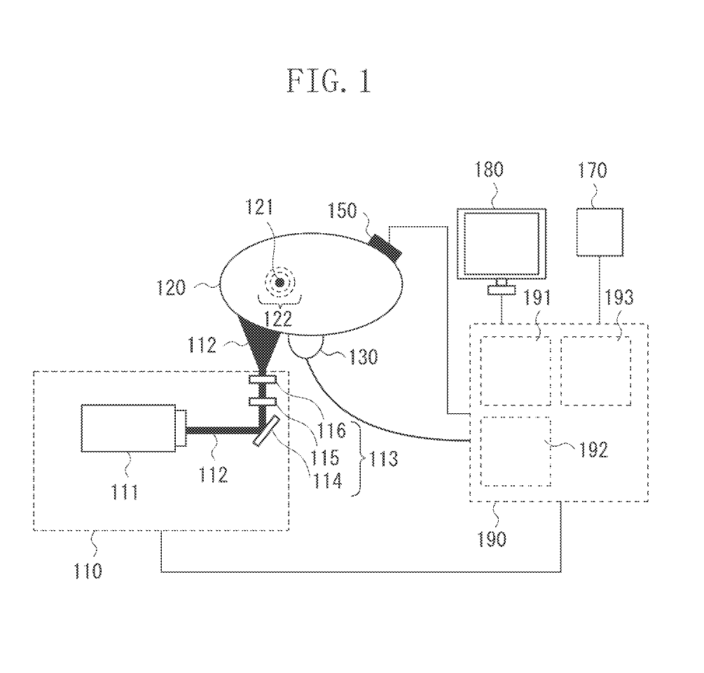

The following describes a basic configuration of a photoacoustic apparatus according to the present exemplary embodiment with reference to FIG. 1.

FIG. 1 is a schematic diagram illustrating the configuration of the photoacoustic apparatus according to the present exemplary embodiment. The photoacoustic apparatus according to the present exemplary embodiment includes a light irradiation unit 110, an acoustic wave reception unit 130, an electrocardiogram acquisition unit 150, an input unit 170, a display unit 180, and a processing unit 190. The light irradiation unit 110 includes a light source 111 and an optical system 113. Detailed configuration thereof will be described below.

First, pulsed light 112 from the light source 111 is guided by the optical system 113. The pulsed light 112 emitted from the optical system 113 is radiated onto a subject 120 and reaches an optical absorber 121 in the subject 120. The optical absorber 121 is typically an in vivo blood vessel, especially a substance such as hemoglobin existing in a blood vessel, a tumor, etc. The optical absorber 121 absorbs the energy of the light to generate a photoacoustic wave 122. The generated photoacoustic wave 122 is propagated in the subject and reaches the acoustic wave reception unit 130.

The acoustic wave reception unit 130 receives the photoacoustic wave 122 to output a time-series received signal. The received signals output from the acoustic wave reception unit 130 are sequentially input to the processing unit 190. The foregoing steps are performed in each of a plurality of times of light irradiation to acquire a plurality of time-series received signals corresponding to the plurality of times of light irradiation.

The processing unit 190 generate subject information of the target region using the plurality of input time-series received signals. Then, the processing unit 190 transmits the generated subject information data to the display unit 180 to cause the display unit 180 to display an image and/or a numerical value of the subject information of the target region. The target region may be preset or input by a user via the input unit 170. The target region is set so as to include at least part of the subject 120. Details of a subject information acquisition method will be described below.

Meanwhile, in a case where the optical absorber is hemoglobin, the amount of hemoglobin is small in a region where the amount of blood in a blood vessel is small, so the optical absorption coefficient of the region is comparatively low. Thus, the sound pressure of a generated photoacoustic wave is low according to Equation (1). In other words, the S/N ratio of a received signal of a photoacoustic wave generated in the region where the amount of blood is small is comparatively low. Further, in a case where the amount of blood is extremely small, a received signal of a photoacoustic wave may be buried in noise. Thus, when the photoacoustic apparatus acquires subject information of the target region, the accuracy of acquired subject information of the region where the amount of blood is small may be low.

In view of the foregoing problem, the photoacoustic apparatus according to the present exemplary embodiment includes the electrocardiogram acquisition unit 150 configured to acquire an electrocardiographic signal of the subject 120. Based on a waveform of an electrocardiographic signal acquired by the electrocardiogram acquisition unit 150, the state of the heart of the subject 120 can be estimated, and the blood flow state of the subject 120 can be accordingly estimated. Thus, the processing unit 190 acquires subject information of the target region based on the electrocardiographic signal of the subject 120, without using received signals of photoacoustic waves generated when the amount of blood in the target region is small, among the plurality of received signals corresponding to the plurality of times of light irradiation. In other words, the processing unit 190 acquires subject information of the target region using at least part of the received signals of the photoacoustic waves generated when the amount of blood in the target region is large, among the plurality of received signals corresponding to the plurality of times of light irradiation. In the present exemplary embodiment, the electrocardiogram acquisition unit 150 corresponds to a blood information acquisition unit.

The signals to be used for the acquisition of subject information are extracted in this manner, so that the subject information can be acquired using more received signals of photoacoustic waves with a high S/N ratio that are generated when the amount of blood is large, i.e., when the amount of hemoglobin as an optical absorber is large. Further, according to the present exemplary embodiment, the subject information can be acquired without using received signals of photoacoustic waves with a low S/N ratio that are generated when the amount of blood is small. This allows acquisition of subject information of a target region with high accuracy. Details of signal extraction timings will be described below.

The following describes each component block of the photoacoustic apparatus according to the present exemplary embodiment.

### Light Source 111

The light source 111 is preferably a pulsed light source capable of generating nanosecond- to microsecond-order pulsed light. Specifically, the pulse width is preferably about 1 to 100 nanoseconds. Further, the wavelength is preferably in the range of about 400 nm to about 1600 nm. Especially in the case of high-resolution imaging of a blood vessel in the vicinity of a surface of a living organism, the wavelength of the light is preferably in the visible light range (400 nm to 700 nm, inclusive). On the other hand, in the case of imaging of a deep portion of a living organism, it is preferable to use light having a wavelength (700 nm to 1100 nm, inclusive) that is less likely to be absorbed by background tissues of the living organism. It is, however, also possible to use the terahertz wave, microwave, or radio wave range.

Specifically, the light source 111 is preferably a laser. Further, in the case of the measurement using light of a plurality of wavelengths, a laser capable of emitting variable wavelengths is more preferable. In a case where the subject 120 is to be irradiated with light of a plurality of wavelengths, a plurality of lasers that emit light having different wavelengths from one another can be used by switching a laser emitting light or by causing the lasers to alternately emit light. In the case of using a plurality of lasers, the plurality of lasers is collectively referred to as the light source.

Various lasers can be used such as a solid-state laser, a gas laser, a dye laser, and a semiconductor laser. Especially a pulse laser such as an Nd:YAG laser and an alexandrite laser is preferable. Further, a Ti:sa laser using Nd:YAG laser light as excitation light, or an optical parametric oscillator (OPO) laser may be used. Further, a light-emitting diode, etc. can be used in place of the laser.

### Optical System 113

The optical system 113 transmits the pulsed light 112 from the light source 111 to the subject 120. Optical elements such as a lens, a mirror, and an optical fiber can be used as the optical system 113. Further, the optical system 113 according to the present exemplary embodiment includes an optical mirror 114 for changing the traveling direction of the pulsed light 112, a light adjustment unit 115, and a diffusion plate 116.

In a biological information acquisition apparatus, the subject of which is a breast, etc., a light emitting unit of the optical system 113 preferably emits pulsed light with a beam diameter widened by the diffusion plate 116, etc. On the other hand, in a photoacoustic microscope, the light emitting unit of the optical system 113 preferably includes a lens, etc. and radiates light with a focused beam diameter to increase the resolution.

Further, the optical system 113 can include the light adjustment unit 115 capable of adjusting the amount of attenuation of the pulsed light 112 emitted from the light source 111. Any unit capable of adjusting the amount of attenuation of the pulsed light 112, such as a mechanical shutter and a liquid crystal shutter, can be used as the light adjustment unit 115.

Further, the optical system 113 may be moved with respect to the subject 120 to allow imaging of a wide range of the subject 120.

Further, the light source 111 may directly radiate light onto the subject 120 without using the optical system 113.

### Subject 120

The following describes the subject 120, but the subject 120 does not constitute a part of the photoacoustic apparatus according to the present exemplary embodiment. The main purposes of use of the photoacoustic apparatus according to the present exemplary embodiment are a diagnosis of a malignant tumor, blood vessel disease, etc. of a human or an animal, follow-up observation of chemical treatment, etc. Thus, the subject 120 is assumed to be a living organism, and a specific diagnosis target region is assumed to be a breast, a neck, an abdominal, or the like of a human body or an animal.

Further, the optical absorber 121 in the subject 120 is preferably an optical absorber having a relatively high optical absorption coefficient in the subject 120. For example, in a case where a measurement target is a human body, the optical absorber 121 may be oxyhemoglobin, deoxyhemoglobin, a blood vessel containing a large amount of oxyhemoglobin or deoxyhemoglobin, or a newborn blood vessel formed in the vicinity of a tumor.

### Acoustic Wave Reception Unit 130

The acoustic wave reception unit 130 includes one or more conversion elements and a housing. Any conversion element capable of receiving an acoustic wave and converting the acoustic wave into an electrical signal may be used. Examples of such conversion elements include a piezoelectric element using a piezoelectric phenomenon such as lead zirconate titanate (PZT), a conversion element using resonance of light, and an electrostatic capacitive conversion element such as a capacitive micromachined ultrasonic transducer (CMUT). In a case where the acoustic wave reception unit 130 includes a plurality of conversion elements, the plurality of conversion elements is preferably arrayed on a flat or curved surface, as called 1D array, 1.5D array, 1.75D array, 2D array, etc.

Further, in order to acquire subject information of a wide range, the acoustic wave reception unit 130 is preferably configured to be mechanically moved with respect to the subject 120 by a scanning mechanism (not illustrated). Further, the optical system 113 (irradiation position of the pulsed light 112) and the acoustic wave reception unit 130 are preferably moved in synchronization with each other.

Further, in a case where the acoustic wave reception unit 130 is a handheld acoustic wave reception unit, the acoustic wave reception unit 130 includes a holding unit with which the user holds the acoustic wave reception unit 130. Further, an acoustic lens may be provided on a reception surface of the acoustic wave reception unit 130. Further, the acoustic wave reception unit 130 may include a plurality of conversion elements.

Further, the acoustic wave reception unit 130 may include an amplifier configured to amplify a time-series analog signal output from a conversion element.

### Electrocardiogram Acquisition Unit 150

The electrocardiogram acquisition unit 150 acquires an electrocardiographic signal of the subject 120. Typically, the electrocardiogram acquisition unit 150 includes an induction electrode for extracting an electrocardiographic signal, an amplifier, an analog-to-digital (A/D) converter, etc. For example, an apparatus discussed in Japanese Patent Application Laid-Open No. 2014-128455 or No. 2014-100244 can be used as the electrocardiogram acquisition unit 150. Based on the electrocardiographic signal acquired by the electrocardiogram acquisition unit 150, the state of the heart of the subject 120 can be estimated. Further, based on the state of the heart that is estimated from the electrocardiographic signal, the blood flow in the blood vessel can be estimated.

### Input Unit 170

The input unit 170 receives various types of input from a user (mainly an examiner such as medical staff), and transmits the input information via a system bus to a component such as the processing unit 190. For example, with the input unit 170, the user can set a parameter relating to imaging, input an imaging start instruction, set an observation parameter such as a range and a shape of a target region, and other image processing operations relating to an image.

The input unit 170 includes a mouse, a keyboard, a touch panel, etc. and performs event notification to software such as an operating system (OS) running on a control unit 193, according to a user operation. Further, in the case of a handheld photoacoustic apparatus, the handheld photoacoustic apparatus preferably includes an input unit 170 for inputting a drive instruction of the light irradiation unit 110. As such an input unit 170, a button switch, a foot switch, or the like that is provided to a probe can be employed.

### Display Unit 180

The display unit 180 may be a display such as a liquid crystal display (LCD), a cathode ray tube (CRT), and an organic electroluminescence (EL) display. Instead of being included in the photoacoustic apparatus according to the present exemplary embodiment, the display unit 180 may be prepared as a separate device and connected to the photoacoustic apparatus.

### Processing Unit 190

The processing unit 190 as a computer includes a calculation unit 191, a storage unit 192, and the control unit 193.

The calculation unit 191 collects time-series received analog signals output from the acoustic wave reception unit 130 and performs signal processing such as amplification of the received signals, AD conversion of the received analog signals, and storage of the digitalized received signals. In general, a circuit called “data acquisition system (DAS)” can be used as the calculation unit 191 configured to perform the foregoing processing. Specifically, the calculation unit 191 includes an amplifier configured to amplify a received signal, an AD converter configured to digitalize a received analog signal, etc.

Further, the calculation unit 191 can acquire generated sound pressure information at each position in the subject using the received signal. The generated sound pressure information at each position in the subject is also referred to as an initial sound pressure distribution in the subject. In a case where the photoacoustic apparatus is a photoacoustic tomography apparatus, the calculation unit 191 performs image reconstruction using the acquired received signals to acquire generated sound pressure data corresponding to positions on a two- or three-dimensional space coordinate. The calculation unit 191 can use a publicly-known image reconstruction method such as universal back projection (UBP), filtered back projection (FBP), and a model base method, as an image reconstruction method. Further, the calculation unit 191 can use delay-and-sum processing as an image reconstruction method.

Further, the calculation unit 191 may apply envelope detection to the acquired received signals with respect to time changes, convert amplitude values of the envelope-detected signals of respective optical pulses in a time axis direction into amplitude values in a depth direction of the conversion element, and plot the converted amplitude values in a pointing direction (typically, depth direction) on a space coordinate. The calculation unit 191 performs the foregoing processing for each position of the conversion element, thereby acquiring initial sound pressure distribution data. Use of the foregoing method is preferable especially in a case where the photoacoustic apparatus is a photoacoustic microscope.

A processor such as a central processing unit (CPU) and a graphics processing unit (GPU), or a calculation circuit such as a field programmable gate array (FPGA) chip can be used as the calculation unit 191 configured to perform the processing to acquire generated sound pressure information. The calculation unit 191 may include a single processor or calculation circuit, or a plurality of processors or calculation circuits.

The storage unit 192 can store a received signal having undergone the AD conversion, various types of distribution data, display image data, various types of measurement parameters, etc. Further, each processing to be performed in a subject information acquisition method described below may be stored in the storage unit 192 as a program to be executed by the control unit 193 in the processing unit 190. The storage unit 192 in which the programs are to be stored is a non-transitory recording medium. The storage unit 192 is typically a storage medium such as a first-in-first-out (FIFO) memory, a read-only memory (ROM), a random-access memory (RAM), and a hard disk. The storage unit 192 may include a single storage medium, or a plurality of storage media.

Further, the processing unit 190 includes the control unit 193 for controlling an operation of each component block of the photoacoustic apparatus. The control unit 193 supplies via a bus a necessary control signal and data to each component block of the photoacoustic apparatus. Specifically, the control unit 193 supplies a light emission control signal for instructing the light source 111 to emit light, a reception control signal for the conversion element in the acoustic wave reception unit 130, etc. The control unit 193 is typically a CPU.

The components of the processing unit 190 may be integrated into a single device or may be separate devices. Further, the calculation unit 191 and the control unit 193 may be included in a single device. In other words, the processing unit 190 may include a single device configured to perform the functions of the calculation unit 191 and the control unit 193.

### Subject Information Acquisition Method

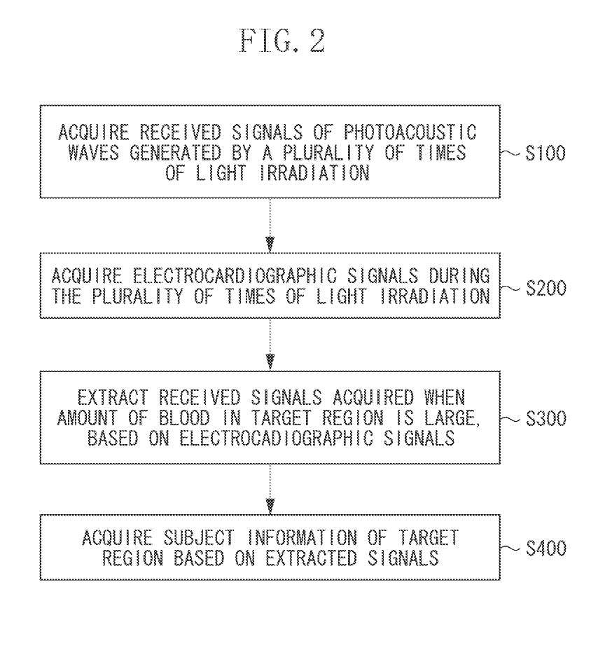

The following describes a flow of the acquisition of subject information performed by the photoacoustic apparatus according to the present exemplary embodiment, with reference to FIG. 2. The control unit 193 reads a program, in which the subject information acquisition method is described, and which is stored in the storage unit 192, and causes the photoacoustic apparatus to execute the following subject information acquisition method.

### (Step S100: Step of Acquiring Received Signals of Photoacoustic Waves Generated by a Plurality of Times of Light Irradiation)

In step S100, the light irradiation unit 110 irradiates the subject 120 with the pulsed light 112. Then, the acoustic wave reception unit 130 receives the photoacoustic wave 122 generated by the irradiation of the pulsed light 112, and outputs a time-series received analog signal. The calculation unit 191 collects the time-series received analog signals output from the acoustic wave reception unit 130, and performs amplification processing on the received signals and AD conversion processing on the received analog signals. Then, the calculation unit 191 stores the digitalized received signals into the storage unit 192. The time-series received signal data stored in the storage unit 192 is also referred to as photoacoustic data. In the present disclosure, the scope of the term “received signal” includes both an analog signal and a digital signal.

Further, in step S100, the light irradiation unit 110 performs a plurality of times of light irradiation, so that a plurality of time-series received signals corresponding to the plurality of times of light irradiation is stored in the storage unit 192.

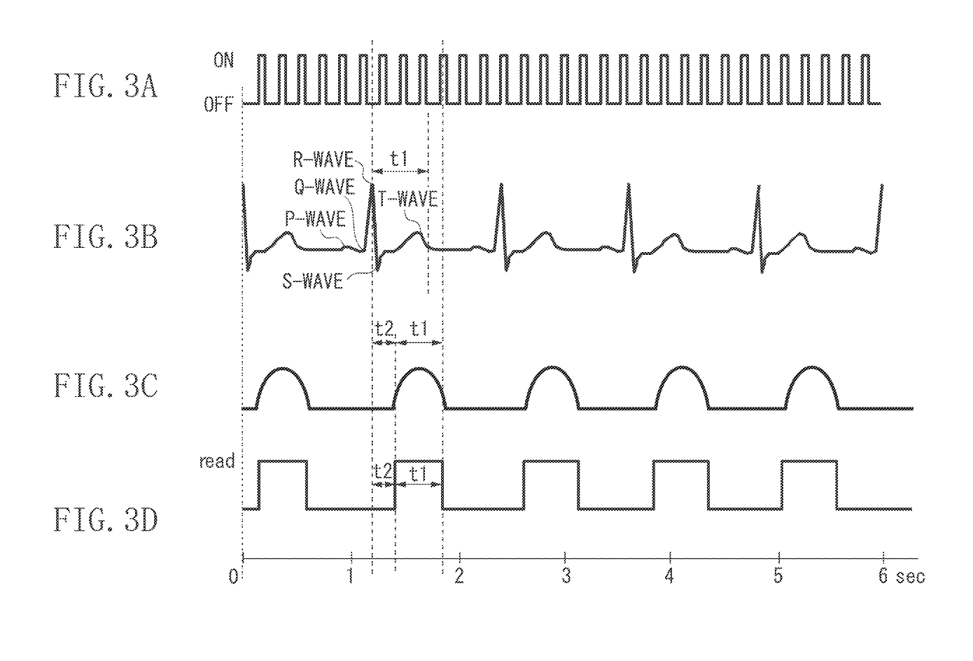

In a case where the light source 111 is a solid-state laser using lamp excitation that easily generates heat, in order to achieve stable driving of the light source 111, it is preferable to emit light at a constant repetition frequency and perform a plurality of times of light irradiation onto the subject 120. FIG. 3A illustrates a driving sequence of the light source 111 according to the present exemplary embodiment. As illustrated in FIG. 3A, in the present exemplary embodiment, the light source 111 emits light at a predetermined repetition frequency (about 5 Hz).

### (Step S200: Step of Acquiring Electrocardiographic Signals During the Plurality of Times of Light Irradiation)

In step S200, the electrocardiogram acquisition unit 150 acquires electrocardiographic signals of the subject 120, and transmits the electrocardiographic signals to the processing unit 190. An electrode included in the electrocardiogram acquisition unit 150 is arranged as appropriate so that an electromyography signal (electrocardiographic signal) relating to the heart can be acquired.

FIG. 3B illustrates a typical waveform of an electrocardiographic signal acquired by the electrocardiogram acquisition unit 150. The electrocardiographic signal illustrated in FIG. 3B has a waveform with a cycle of about 1.2 seconds. Typically, a waveform of an electrocardiographic signal is formed by a combination of a P-wave, a Q-wave, an R-wave, an S-wave, and a T-wave. In general, a period from a peak of an R-wave to the vicinity of an end of a T-wave corresponds to a ventricular systole phase, and blood is pumped to an artery. Further, a period from the vicinity of an end of a T-wave to a peak of an R-wave corresponds to a ventricular dilatation phase. As used herein, a time t1 from a peak of an R-wave to the vicinity of an end of a T-wave will be referred to as “first time from a generation timing of the R-wave to a generation timing of the T-wave.”

FIG. 3C is a graph showing a change in the amount of blood in the target region. As described above, as to the state of the heart, triggered by the R-wave of the electrocardiographic signal, ventricular systole is started, and blood is accordingly started to be pumped into arteries. However, as can be understood from FIG. 3C, the amount of blood in the target region increases not at the generation timing of the R-wave, but with a time lag of a time t2 from the generation timing of the R-wave to the timing at which the blood flow corresponding to the ventricular systole reaches the target region. Then, the period in which the amount of blood is large is considered to be maintained for the time t1 of the ventricular systole period, after the time t2 elapses from the generation timing of the R-wave.

As used herein, the time t2 from the generation timing of the R-wave to the timing at which the blood flow corresponding to the ventricular systole reaches the target region is also referred to as “delay time.”

### (Step S300: Step of Extracting Received Signals Acquired When the Amount of Blood in Target Region is Large, Based on Electrocardiographic Signals)

In step S300, the calculation unit 191 as a subject information acquisition unit extracts signals to be used for the acquisition of subject information, from the plurality of time-series received signals corresponding to the plurality of times of light irradiation that are acquired in step S100, based on the electrocardiographic signals acquired in step S200.

Based on the electrocardiographic signals acquired by the electrocardiogram acquisition unit 150, the calculation unit 191 determines a timing at which the amount of blood in the target region is large. Then, the calculation unit 191 reads from the storage unit 192 the received signals of the photoacoustic waves generated at the timing. On the other hand, the calculation unit 191 does not read from the storage unit 192 received signals of photoacoustic waves generated at a timing at which the amount of blood in the target region is small, and the received signals generated at the timing are not used for the acquisition of subject information.

FIG. 3D illustrates a signal extraction sequence performed by the calculation unit 191, and indicates that the calculation unit 191 extracts the received signals acquired at “read.” The calculation unit 191 extracts, among the plurality of time-series received signals acquired in step S100, the received signals of the photoacoustic waves generated in a period from a timing at which the delay time t2 elapses from the generation timing of the R-wave of the electrocardiographic signal, to a timing at which the time t1 of the ventricular systole elapses. In other words, the calculation unit 191 reads from the storage unit 192 the received signals of the photoacoustic waves generated by the light irradiation performed in a period in which a blood flow caused by electric stimulation exists in the target region, i.e., a period in which the amount of blood is large. On the other hand, the calculation unit 191 does not read from the storage unit 192 the received signals of the photoacoustic waves generated by the light irradiation performed in a period other than the period in which the amount of blood is large.

The signals extracted in step S300 are the received signals of the photoacoustic waves generated at a timing at which the amount of blood is increased due to the ventricular systole. Thus, the extracted signals include many signals with a high S/N ratio.

Since the speed of light is much more faster than the speed of a photoacoustic wave, it can be considered that the photoacoustic waves are simultaneously generated at the respective positions in the target region at the timing at which the subject 120 is irradiated with the pulsed light 112. In the present specification, the timing at which the subject 120 is irradiated with the pulsed light 112 is referred to as the timing at which photoacoustic waves are generated by the pulsed light 112.

Further, it is known that typically the time t1 from the generation timing of the R-wave to the generation timing of the T-wave is 0.3 seconds or longer and 0.45 seconds or shorter. Thus, the calculation unit 191 may read from the storage unit 192 the received signals of the photoacoustic waves generated in a period in which the amount of blood is large, i.e., in a predetermined time of 0.3 seconds or longer and 0.45 seconds or shorter after the time t2 elapses from the generation timing of the R-wave.

Meanwhile, the calculation unit 191 can detect from the electrocardiographic signal a timing at which a specific wave such as the R-wave and the T-wave is generated. For example, the calculation unit 191 can detect, as the R-wave, a wave of an electrocardiographic signal having an amplitude greater than a predetermined amplitude. Further, for example, the calculation unit 191 can perform template matching of the electrocardiographic signals with template waveforms of the R-wave and the T-wave that are stored in the storage unit 192, and a wave having a high similarity can be detected as the R-wave or the T-wave. A method for detecting a specific wave may be any method as long as a characteristic waveform such as the R-wave and the T-wave can be detected.

The blood flow corresponding to the ventricular systole reaches the target region after the elapse of a time corresponding to a value obtained by dividing the length of the blood vessel between the heart and the target region by the blood flow velocity, since the R-wave has been generated. Thus, the calculation unit 191 can determine an extraction start timing of signals to be used for the acquisition of subject information, based on the generation timing of the R-wave, information on the length of the blood vessel between the heart and the target region, and information on the blood flow velocity. However, in order to determine the extraction start timing of signals to be used, in the above-described manner, it is necessary to measure for each subject the distance between the heart and the target region and the blood flow velocity. This may lead to an increase in apparatus size.

Thus, it is preferable to select the extraction start timing from those determined in advance for different target regions corresponding to different portions of the subject. More specifically, the storage unit 192 preferably includes a relation table indicating a relation between the type of a target region and the delay time t2. Further, the photoacoustic apparatus preferably includes the input unit 170 configured to allow the user to input the type of a target region. For example, the input unit 170 can be configured to allow the user to select the type of a target region from a plurality of types, or portions of the subject, displayed on the display unit 180. Then, the calculation unit 191 can read out, from the relation table stored in the storage unit 192, the delay time t2 corresponding to the type input via the input unit 170. The calculation unit 191 detects from the electrocardiographic signal the generation timing of the R-wave, and can extract a desired signal from the received signals of the photoacoustic waves generated after the delay time t2 read from the storage unit 192 elapses from the detected generation timing.

While the foregoing describes the type of the target region as the information necessary for the determination of the delay time t2, the information necessary for the determination of the delay time t2 is not limited to the type of the target region. For example, it is considered that even if the type of the target region is the same, the delay time t2 may vary depending on, for example, the age of the subject. Thus, the input unit 170 is preferably configured to allow input of information such as the age of the subject, etc. in addition to the type of the target region. In other words, the input unit 170 is preferably configured to allow input of at least the type of the target region. Further, the control unit 193 preferably reads, from the relation table, the delay time t2 corresponding to the input information such as the age of the subject.

Further, in a case where the target region of the photoacoustic apparatus is predetermined, the storage unit 192 preferably stores information on the delay time t2 obtained in advance. Then, the calculation unit 191 can detect from the electrocardiographic signal the generation timing of the R-wave, and extract a desired signal from the received signals of the photoacoustic waves generated after the delay time t2 stored in the storage unit 192 elapses from the detected generation timing.

In a case where the period from the generation timing of the R-wave to the timing at which the blood flow corresponding to the ventricular systole reaches the target region can be ignored, the generation timing of the R-wave may be used as the extraction start timing. In other words, in this case, the delay time t2 may be t2=0.

In the present exemplary embodiment, the received signal extraction timing is set based on the assumption that the amount of blood increases during the time t1 of the ventricular systole phase after the delay time t2 elapses from the generation timing of the R-wave. The extraction timing setting, however, is not limited to this. For example, the time t1 corresponds to the time of the ventricular systole phase, and there may be a case where most of the pumping of the blood is completed before the time t1 elapses, depending on the amount of stored blood. In other words, there may be a case where the time of the ventricular systole phase does not match the time needed for pumping the blood. In this case, the amount of blood may increase only for a period shorter than the time t1. In this case, the processing unit 190 preferably acquires subject information using at least part of the received signals of the photoacoustic waves generated before the time t1 elapses after the delay time t2 elapses from the generation timing of the R-wave. More specifically, the processing unit 190 preferably uses many received signals acquired before a half of the time t1 elapses, among the received signals acquired before the voltage application time t1 elapses after the delay time t2 elapses from the generation timing of the R-wave.

Further, for example, even during the time t1 after the delay time t2 elapses from the generation timing of the R-wave, at a timing at which the increase of the amount of blood is not large enough, a received signal to be acquired may not have a sufficiently high S/N ratio. Thus, the calculation unit 191 preferably extracts a received signal having a larger amplitude than a predetermined value, during the voltage application time t1 after the delay time t2 elapses from the generation timing of the R-wave. In this way, a received signal with an especially high S/N ratio can selectively be extracted in the period in which the amount of blood increases.

The sequence illustrated in FIG. 3D may be executed in real time in parallel with the sequences illustrated in FIGS. 3A to 3C, or may be executed after the sequences illustrated in FIGS. 3A to 3C are completed in all the periods.

In the present exemplary embodiment, a desired signal is extracted from the plurality of time-series received signals stored in the storage unit 192. This, however, is not a limiting example, and any method can be used as long as subject information can be acquired by selectively using a desired signal. For example, among the analog electrical signals output from the acoustic wave reception unit 130, the analog electrical signals corresponding to the received signals of the photoacoustic waves generated when the amount of blood is small may be excluded from the signals to be stored in the storage unit 192. Consequently, the received signals of the photoacoustic waves generated when the amount of blood is large are selectively stored in the storage unit 192. Then, the calculation unit 191 may acquire subject information by selectively using the received signals of the photoacoustic waves generated when the amount of blood is large that are stored in the storage unit 192.

### (Step S400: Step of Acquiring Subject Information of Target Region Based on Extracted Received Signals)

In step S400, the calculation unit 191 acquires subject information of the target region based on the received signals extracted in step S300. In the present exemplary embodiment, the calculation unit 191 calculates generated sound pressure information of the photoacoustic wave at each position in the target region, i.e., initial sound pressure distribution, as subject information, and stores the subject information into the storage unit 192.

The initial sound pressure distribution acquired in step S400 is calculated based on the signals with a high S/N ratio that are extracted in step S300, so the accuracy is high. Thus, if the calculation unit 191 causes the display unit 180 to display an image of the initial sound pressure distribution stored in the storage unit 192, an image with high image quality such as resolution and contrast can be provided to the user.

The calculation unit 191 may calculate the optical fluence, i.e., light amount distribution of the pulsed light 112 having reached each position in the target region. In the present exemplary embodiment, the calculation unit 191 can acquire information on the light amount distribution of the pulsed light 112 in the target region by solving a light diffusion equation discussed in Bin Luo and Sailing He, Optics Express, Vol. 15, Issue 10, pp. 5905-5918 (2007), and can store the acquired information in the storage unit 192. The calculation unit 191 may acquire the light amount distribution using any method as long as the light amount distribution in the target region can be acquired.

Then, the calculation unit 191 may acquire, as subject information, the optical absorption coefficient distribution in the target region according to Equation (1) using the initial sound pressure distribution and the light amount distribution in the target region that are stored in the storage unit 192.

In step S400, the calculation unit 191 may acquire subject information of one frame from the time-series received signal acquired by one-pulsed light irradiation, among the signals extracted in step S300. Further, the calculation unit 191 may acquire subject information of one frame from the plurality of time-series received signals acquired by a plurality of times of light irradiation, among the signals extracted in step S300. In other words, the calculation unit 191 is only required to acquire subject information using at least part of the received signals of the photoacoustic waves generated when the amount of blood is large.

According to the foregoing subject information acquisition method, it is possible to suppress the influence of the amount of blood in the blood vessel on the accuracy of acquired subject information.

Further, the photoacoustic apparatus according to the present exemplary embodiment may perform the foregoing steps using light having different wavelengths to similarly acquire optical absorption coefficient distributions. Then, the calculation unit 191 can acquire, as subject information, the concentration distribution information of substances included in the subject 120, using the plurality of optical absorption coefficient distributions corresponding to the light having different wavelengths from one another.

However, in a case where a single light source is used for generating light of a plurality of wavelengths, it may take time to switch the wavelengths. If the wavelengths are switched when the amount of blood is large, the number of times the light irradiation can be performed while the amount of blood is large decreases to result in lower accuracy of subject information. Accordingly, it is preferable to switch the wavelengths when the amount of blood is small. For example, the light irradiation unit 110 irradiates the subject 120 with light of a first wavelength λ1 during one cycle of an electrocardiographic signal, where one cycle is from an R-wave to the next R-wave. Then, in a period in which the amount of blood is small in the cycle, a wavelength changing mechanism in the light source 111 is driven so that the light source 111 becomes ready to generate light of a second wavelength λ2. Then, in the next cycle, the light irradiation unit 110 irradiates the subject 120 with light of the second wavelength λ2.

In this way, the wavelengths can be switched in the period in which the amount of blood is small and the received signals of which are determined not to be used. This allows efficient irradiation of the subject 120 with light of a plurality of wavelengths while the amount of blood is large, without decreasing the number of times the light irradiation can be performed in the period in which the amount of blood is large and the received signals of which are determined to be used. Further, in the present exemplary embodiment, signals that can be extracted for the acquisition of subject information can be ensured efficiently, so that the accuracy of the subject information acquisition can be increased efficiently.

As described above, in the present exemplary embodiment, subject information is acquired based on a plurality of received signals acquired in a common period in each fluctuation cycle of the amount of blood that is repeated a plurality of times in association with a pulse of the subject. The subject information is acquired from the received signals of the same timings. This can suppress the influence of a change in the amount of blood on the subject information to be acquired. Especially, in the present exemplary embodiment, the received signals acquired in a period in which the amount of blood is large in the fluctuation cycle are used, so that an image with high accuracy of subject information can be acquired.

The following describes a second exemplary embodiment.

In the first exemplary embodiment, subject information is generated only from the received signals acquired in a period in which the amount of blood is large. However, a user may desire to observe a subject region in a period in which the amount of blood is small. Thus, in the present exemplary embodiment, the description will be given of a case in which received signals acquired in a period in which the amount of blood is small, which are not used for the acquisition of subject information in the first exemplary embodiment, are acquired and subject information is acquired based on the acquired received signals.

A photoacoustic apparatus according to the present exemplary embodiment has a similar configuration to that described in the first exemplary embodiment.

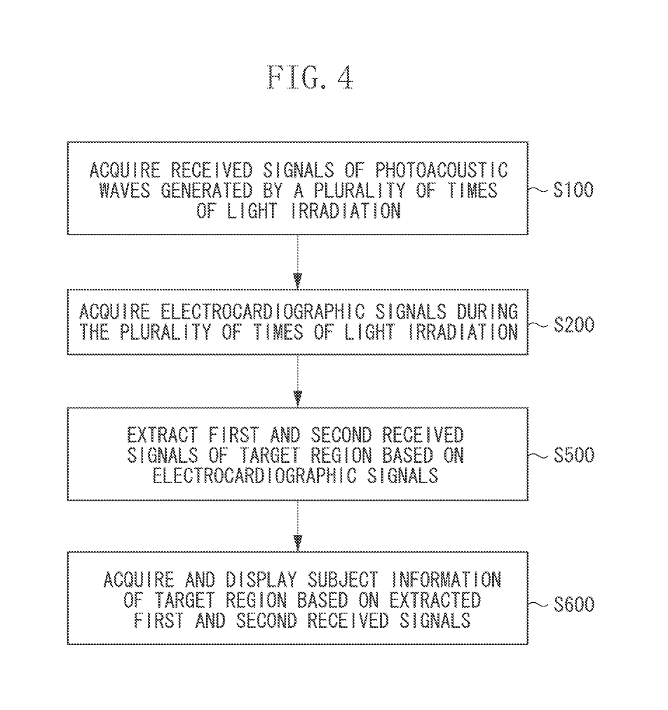

The following describes a flow of acquisition of subject information by the photoacoustic apparatus according to the present exemplary embodiment, with reference to FIG. 4. Steps S100 and S200 are similar to those in the first exemplary embodiment.

### (Step S500: Step of Extracting Received Signal Acquired When the Amount of Blood in Target Region is Large as First Received Signal and Received Signal Acquired at Another Timing as Second Received Signal, Based on Electrocardiographic Signals)

In step S500, the calculation unit 191 as the subject information acquisition unit extracts signals to be used for the acquisition of subject information, from the plurality of time-series received signals corresponding to the plurality of times of light irradiation that are acquired in step S100, based on the electrocardiographic signals acquired in step S200.

The calculation unit 191 estimates a period in which the amount of blood in the target region is large, based on the electrocardiographic signals acquired by the electrocardiogram acquisition unit 150. Then, the calculation unit 191 reads from the storage unit 192 a received signal of a photoacoustic wave generated in the period as a first received signal. On the other hand, the calculation unit 191 reads from the storage unit 192 a received signal of a photoacoustic wave generated at a timing at which the amount of blood in the target region is small as a second received signal. In other words, the calculation unit 191 extracts a received signal acquired at “read” in FIG. 3D as a first received signal, and extracts a received signal acquired at a timing other than “read” as a second received signal. The received signals extracted as different types in this manner are each referred to as a received signal group.

The signals extracted in step S500 reads, as different received signals, a photoacoustic wave generated at a timing at which the amount of blood is increased due to ventricular systole and a photoacoustic wave generated at a timing at which the amount of blood is decreased due to ventricular dilatation. Thus, a fluctuation in the amount of blood at the respective timings at which the signals are generated is small in each of the received signal groups.

### (Step S600: Step of Acquiring and Displaying Subject Information of Target Region Based on Extracted First and Second Received Signals)

In step S600, the calculation unit 191 acquires subject information of the target region based on each of the first and second received signals extracted in step S500. In the present exemplary embodiment, the calculation unit 191 calculates generated sound pressure information of the photoacoustic wave at each position in the target region, i.e., initial sound pressure distribution, as subject information and stores the subject information in the storage unit 192.

As described above in the first exemplary embodiment, the subject information is not limited to the initial sound pressure distribution, and may be optical absorption coefficient distribution information or concentration distribution information of substances included in the subject 120.

There is a known method of averaging numerical values of the optical absorption coefficient distribution or the substance concentration distribution that are acquired from a plurality of times of laser irradiation, to increase the S/N ratio of the numerical values. Meanwhile, the optical absorption coefficient distribution and the substance concentration distribution are proportional to the amount of hemoglobin, so it can be said that the optical absorption coefficient distribution and the substance concentration distribution are proportional to the amount of blood. Thus, subject information with a smaller fluctuation in the amount of blood can be acquired from an average of subject information acquired from the first received signal and an average of subject information acquired from the second received signal, compared to an average of subject information acquired from a plurality of received signals acquired irrespective of the amount of blood in the blood vessel.

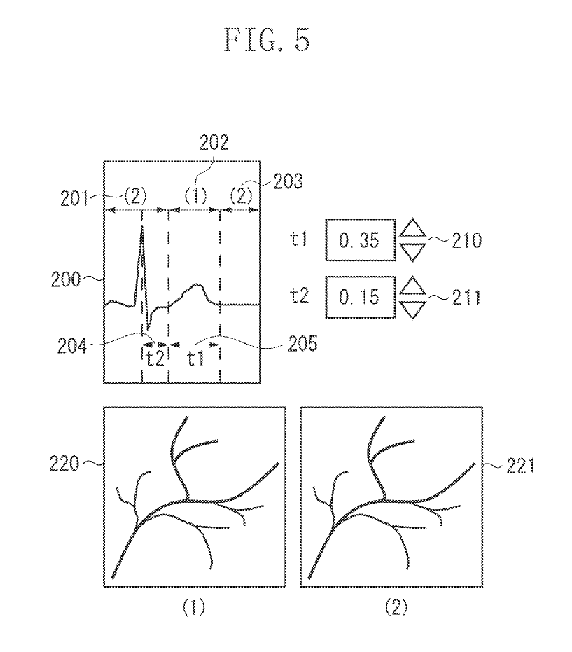

The following describes a method of displaying the acquired subject information, with reference to FIG. 5.

A region 220 is a region where an image of the initial sound pressure distribution generated from the first received signal is displayed. A region 221 is a region where an image of the initial sound pressure distribution generated from the second received signal is displayed. In general, an initial sound pressure distribution is acquired as a three-dimensional (3D) image. Thus, as to the display of the initial sound pressure distribution, a 3D image may be displayed, or a cross section of the 3D image, a maximum intensity projection (MIP) image of a range, etc. may be displayed.

A region 200 is a region where a range in which a received signal is extracted as the first received signal and a range in which a received signal is extracted as the second received signal are displayed. A range 204 is a range indicating the time t2 described in the first exemplary embodiment. A range 205 is a range indicating the time t1 described in the first exemplary embodiment. A range 201 indicates a period from a start of the P-wave to a timing at which the time t2 elapses from a peak of the R-wave, and is a range in which a received signal is extracted as the second received signal. A range 202 indicates the time t1, and is a range in which a received signal is extracted as the first received signal. A range 203 indicates a range from an end of the time t1 to a start of the P-wave, and is a range in which a received signal is extracted as the second received signal. A typical electrocardiographic waveform may be displayed in a background of the region 200 to allow the user to understand with ease. Further, the latest electrocardiographic waveform actually acquired by the electrocardiogram acquisition unit 150 may be displayed. The typical electrocardiographic waveform and the latest electrocardiographic waveform may be displayed alongside of the region 200.

An item 210 is a user interface (UI) part with which the time t1 can be input/changed. An item 211 is a UI part with which the time t2 can be input/changed. The widths of the ranges 205 and 204 may be changed in conjunction with changes made with the items 210 and 211. Further, the first and second received signals extraction processing of step S500 is executed again based on the times t1 and t2 newly set in conjunction with changes in the items 210 and 211. Further, subject information is acquired again, and images to be displayed on the regions 220 and 221 are updated. In the example illustrated in FIG. 5, if the user selects an upper or lower triangle indicated as the item 210, the length of the time t1 indicated in a text box is updated, and the distance between dashed lines defining the time t1 is changed on the region 200. The user may set the range of the time t1 by directly inputting numbers into the text box or by dragging a dashed line in the region 200 with the mouse, etc. to determine a start time and an end time of the time t1. The time t2 can be set using a similar method to that used for the time t1.

The subject information to be displayed is not limited to the initial sound pressure distribution and may be the optical absorption coefficient distribution or the substance concentration distribution.

In the present exemplary embodiment, the description has been given of an example case where one cycle of the electrocardiographic waveform is divided into two periods, one of which is a period in which the amount of blood in a subject region is large (period (1) in FIG. 5), and the other one of which is a period in which the amount of blood is small (period (2) in FIG. 5). Alternatively, one cycle may be divided into three or more segments. If one cycle is divided into a larger number of segments, the fluctuation in the amount of blood in each segment becomes smaller. Thus, received signals of more uniform signal level can be acquired. On the other hand, if the number of divisions of the electrocardiographic waveform is increased, the number of received signals acquired in each segment decreases, so the effect of increasing the S/N ratio that is produced by averaging the received signals decreases. It is desirable to take this trade-off into consideration to determine the number of divisions into which one cycle is to be divided.

While an image based on the first received signal and an image based on the second received signal are displayed alongside of each other in the present exemplary embodiment, any other display method may be used. For example, two images may be superimposed and displayed. Further, the images may be displayed alternately. If the number of divisions of the electrocardiographic waveform is increased, acquired images can be displayed like a moving image. Since images of averages of the received signals acquired by the plurality of times of irradiation are sequentially displayed, a moving image based on images with a high S/N ratio can be provided to the user, compared to a case where the initial sound pressure distributions each acquired from the received signal acquired by a single time of laser irradiation are sequentially displayed like a moving image.

While subject information is acquired and displayed after the acquisition of the photoacoustic wave signal and the electrocardiographic signal in the present exemplary embodiment, subject information may be acquired and displayed in real time while the photoacoustic wave signal and the electrocardiographic signal are acquired. Further, in a case where subject information is acquired and displayed in real time, instead of using all the photoacoustic wave signals and electrocardiographic signals acquired from the start of acquisition, only part of the photoacoustic wave signals and electrocardiographic signals may be used. For example, it can be considered to use photoacoustic wave signals and electrocardiographic signals acquired during a period from a time anterior to a current time by a given time to the current time. In this way, even if the subject is moved, more accurate subject information can be acquired.

The second exemplary embodiment described above produces a similar advantage to that of the first exemplary embodiment. Further, according to the present exemplary embodiment, subject information of each of different periods in a fluctuation cycle of the amount of blood can be provided to the user. An artery pulses at a timing at which the amount of blood increases, and it is considered that an artery and a vein can be distinguished from each other by comparing the plurality of pieces of subject information.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the foregoing describes the specific exemplary embodiments of the present invention, it should be understood that the foregoing specific exemplary embodiments are not intended to limit the scope of the invention and can be modified within the technical idea of the present invention.

For example, while the foregoing describes the case where an electrocardiograph is used as the blood information acquisition unit configured to determine an increase/decrease in the amount of blood in a measured target, the amount of blood in a measured target may be determined using any other method for measuring a pulse of a subject. For example, an infrared pulsimeter may be used.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application 2014-242454, filed Nov. 28, 2014, and No. 2015-204123, filed Oct. 15, 2015, which are hereby incorporated by reference herein in their entirety.

What is claimed is:

1. A photoacoustic apparatus comprising:
a light irradiation unit configured to irradiate a subject with pulsed light a plurality of times;
a reception unit configured to receive photoacoustic waves generated by the plurality of times of irradiation of the subject with the pulsed light from the light irradiation unit, and output a plurality of signals corresponding to the plurality of times of light irradiation;
a blood information acquisition unit configured to acquire information on an amount of blood of the subject; and
a subject information acquisition unit configured to acquire subject information of a target region in the subject based on the plurality of signals,
wherein the subject information acquisition unit acquires the subject information based on a plurality of signals acquired in a common period in each of repeated cycles of fluctuation of the amount of blood.

2. The photoacoustic apparatus according to claim 1, wherein the subject information acquisition unit causes a display unit to display the subject information.

3. The photoacoustic apparatus according to claim 1, wherein the subject information acquisition unit acquires first subject information based on a plurality of signals acquired in a first period in each of the cycles, and acquires second subject information based on a plurality of signals acquired in a second period different from the first period in each of the cycles.

4. The photoacoustic apparatus according to claim 3, wherein the subject information acquisition unit causes a display unit to display the first subject information and the second subject information alongside of each other.

5. The photoacoustic apparatus according to claim 4, wherein the subject information acquisition unit causes the display unit to display the first subject information and the second subject information so that the first subject information and the second subject information are superimposed.

6. The photoacoustic apparatus according to claim 2, wherein the subject information acquisition unit causes the display unit to display information indicating the common period in each of the cycles.

7. The photoacoustic apparatus according to claim 2, wherein the blood information acquisition unit includes an electrocardiogram acquisition unit configured to acquire an electrocardiographic signal of the subject, and
wherein the subject information acquisition unit causes the display unit to display the electrocardiographic signal.

8. The photoacoustic apparatus according to claim 1, wherein the blood information acquisition unit includes an electrocardiogram acquisition unit configured to acquire an electrocardiographic signal of the subject, and
wherein the subject information acquisition unit acquires the subject information using at least part of signals corresponding to photoacoustic waves generated in a period from a start timing determined based on a generation timing of an R-wave of the electrocardiographic signal until a first time from the generation timing of the R-wave of the electrocardiographic signal to a generation timing of a T-wave of the electrocardiographic signal elapses, and without using a signal corresponding to a photoacoustic wave generated in a period other than the period from the start timing until the first time elapses.

9. The photoacoustic apparatus according to claim 8, wherein the subject information acquisition unit determines, based on the electrocardiographic signal, the generation timing of the R-wave of the electrocardiographic signal and the generation timing of the T-wave of the electrocardiographic signal.

10. The photoacoustic apparatus according to claim 8, wherein the subject information acquisition unit acquires the subject information using at least part of signals corresponding to photoacoustic waves generated in a period from the start timing determined based on the generation timing of the R-wave of the electrocardiographic signal until a time of 0.3 seconds or longer and 0.45 seconds or shorter elapses.

11. The photoacoustic apparatus according to claim 8, further comprising:
an input unit configured to allow input of a type of the target region; and
a storage unit configured to store a relation table of the type of the target region and a second time from the generation timing of the R-wave to the start timing,
wherein the subject information acquisition unit reads out from the relation table the second time corresponding to the type of the target region that is input via the input unit, and
further wherein the subject information acquisition unit sets the start timing based on the generation timing of the R-wave of the electrocardiographic signal and the second time read from the relation table.

12. The photoacoustic apparatus according to claim 8, wherein the subject information acquisition unit sets the start timing based on the generation timing of the R-wave of the electrocardiographic signal, information on a distance between a heart of the subject and the target region, and information on a blood flow velocity.

13. The photoacoustic apparatus according to claim 8, wherein the light irradiation unit irradiates the subject with pulsed light a plurality of times at a constant repetition frequency.

14. The photoacoustic apparatus according to claim 8, wherein the light irradiation unit includes a light source capable of emitting pulsed light with a plurality of wavelengths different from one another, and
wherein the light source switches the plurality of wavelengths in a period other than the period from the start timing until the first time elapses.

15. The photoacoustic apparatus according to claim 8, further comprising a storage unit configured to store the plurality of signals,
wherein the subject information acquisition unit extracts, from among the plurality of signals stored in the storage unit, at least part of the signals corresponding to the photoacoustic waves generated in the period from the start timing until the first time elapses, and acquires the subject information based on the extracted signal.

16. A subject information acquisition method of acquiring subject information based on a plurality of signals corresponding to a plurality of times of light irradiation and acquired by receiving photoacoustic waves generated by the plurality of times of irradiation of a subject with pulsed light, the method comprising:
acquiring the subject information based on a plurality of signals acquired in a common period in each of repeated cycles of fluctuation of an amount of blood.

17. The subject information acquisition method according to claim 16, wherein first subject information based on a plurality of signals acquired in a first period in each of the cycles is acquired, and second subject information based on a plurality of signals acquired in a second period different from the first period in each of the cycles is acquired.

18. The subject information acquisition method according to claim 16, wherein the subject information is acquired by selectively using, from among the plurality of signals, at least part of signals corresponding to photoacoustic waves generated in a period from a start timing determined based on a generation timing of an R-wave of an electrocardiographic signal acquired from the subject until a first period from the generation timing of the R-wave of the electrocardiographic signal to a generation timing of a T-wave of the electrocardiographic signal elapses.

19. A non-transitory memory storing a program for causing a computer to execute the subject information acquisition method according to claim 16.

20. A photoacoustic apparatus comprising:
a light irradiation unit configured to irradiate a subject with pulsed light;
a reception unit configured to receive a photoacoustic wave generated by the irradiation of the subject with the pulsed light from the light irradiation unit, and output a signal;
a subject information acquisition unit configured to acquire subject information based on the signal; and
a blood flow information acquisition unit configured to acquire blood flow information of the subject,
wherein the subject information acquisition unit acquires the subject information based on the blood flow information without using a signal based on a photoacoustic wave generated in a period in which a blood flow is relatively low in a cycle of the blood flow fluctuation that is repeated in a target region of the subject.
